(19) United States
(12) Patent Application Publication
(10) Pub. No.: US 2017/0265751 A1
(43) Pub. Date: Sep. 21, 2017

(54) NON-CONTACT LASER ULTRASOUND SYSTEM

(71) Applicant: Massachusetts Institute of Technology, Cambridge, MA (US)

(72) Inventors: Robert W. Haupt, Lexington, MA (US); Anthony Samir, Newton, MA (US); Charles M. Wynn, Groton, MA (US); Jae H. Kyung, Arlington, MA (US); Kevin W. Holman, Wilmington, MA (US)

(21) Appl. No.: 15/456,842

(22) Filed: Mar. 13, 2017

Related U.S. Application Data
(60) Provisional application No. 62/309,497, filed on Mar. 17, 2016.

(51) Int. Cl. A61B 5/00; A61B 8/08; G01H 9/00

(52) U.S. Cl. CPC A61B 5/0095; G01H 9/00; A61B 8/485

(57) ABSTRACT

Non-contact ultrasound imaging system. The system includes a pulsed near infrared scanning laser source for illuminating a surface of a structure to generate ultrasonic elastic waves that propagate into the structure. A laser Doppler vibrometer measures vibration of the surface caused by the propagating ultrasonic waves in the structure and a data acquisition module processes data from the vibrometer to construct an image of the structure.

This application claims priority to U.S. Provisional Application Ser. No. 62/309497 filed on Mar. 17, 2016, the contents of which are incorporated herein by reference in their entirety.

“This invention was made with Government support under Contract No. FA8721-05-C-0002 awarded by the U.S. Air Force. The Government has certain rights in the invention.”

### BACKGROUND OF THE INVENTION

This invention relates to ultrasound imaging technology and, more particularly, to an ultrasound system that eliminates contact of a probe with the surface of a body.

Ultrasound is an ideal imaging modality used in medical practice: it is portable, inexpensive, has no known bio-effects, and produces images with excellent spatial and temporal resolution. Despite these advantages, MRI and CT are the dominant modalities used for most medical imaging requirements while accepting high cost and imposed health risks. Ultrasound is typically acquired with hand held transducers that depend on the operator's applied pressure and selection of angle planes with no fixed acquisition frame. These factors lead to inter-operator variability that introduces significant image distortion and limits image accuracy and confidence. For example, the American Thyroid Association guidelines require an increase in thyroid nodule volume of more than 50%, or an increase of more than 20% in two or more measured dimensions, prior to recommending biopsy when using conventional ultrasound. This is because interoperator variability is so high, large increases in nodule size are necessary before nodule growth can be confidently diagnosed. The European Organization for Research and Treatment of Cancer (EORTC) Response Evaluation Criteria In Solid Tumors (RECIST) guidelines do not permit the use of ultrasound tumor measurements for the follow-up of cancer, owing to high interobserver variability. This means that patients with cancer must be followed up with CT scans (high cost and ionizing radiation) or MRI scans (high cost).

Further, standardized contact transducer systems can cause patient risk, discomfort or pain in cases involving trauma, damaged or burned skin, bleeding, infection-susceptible regions, delicate surgical configurations or for difficult-to-reach areas.

An object of the invention is to mitigate ultrasound inter-operator and operator variability, and possibly patient discomfort, using a standoff optical concept that generates and measures ultrasonic waves without patient contact.

### SUMMARY OF THE INVENTION

The non-contact, ultrasound imaging system according to the invention includes a pulsed near infrared scanning laser source for illuminating a surface of a structure to generate ultrasonic elastic waves that propagate into the structure. A laser Doppler vibrometer is provided to measure vibration of the surface resulting from the propagating ultrasonic waves in the structure. A data acquisition module processes data from the vibrometer to construct an image of the structure. The structure is often a human body.

In a preferred embodiment, the system further includes a camera to record locations of the illuminating and vibrometer beam locations. A suitable pulsed near infrared laser is a Q-switched laser. The Q-switched laser may operate at a 1-10 kilohertz framerate. In another preferred embodiment, the structure is a biological entity and the surface is skin. The system components may be supported by an x-y translation stage mounted on a stand that fits over the structure. A suitable laser wavelength for illumination is 1550 nm.

### BRIEF DESCRIPTION OF THE DRAWING

FIG. 1 is a schematic illustration of an embodiment the ultrasound imaging system disclosed herein.

FIG. 2 is a schematic illustration showing the creation of ultrasonic waves and measuring vibrating skin surface.

FIG. 3 includes ultrasound images of a foreign body embedded in a muscle tissue.

FIG. 4 is a schematic illustration of the system of the invention showing it mounted on a frame that fits over a subject being probed.

### DESCRIPTION OF THE PREFERRED EMBODIMENT

A pulsed near IR source (Q-switched laser) converts optical energy to ultrasonic waves within the skin surface via photoacoustic (PA) mechanisms. Laser Doppler vibrometry (LDV) is then used to measure probing ultrasound signals that return to the skin surface. A short wavelength (SWIR) or visible spectrum camera is used to record the image of the excitation and LDV beam locations on the patient target. A frame rate of 1-10 kiloHertz is adequate to capture relevant patient motion and jitter during the ultrasonic data acquisition process. A lidar system or optical camera set can also be used to map the 3D patient surface topography and then used to correct for transmitter and receiver geometries when applied to data and image processing.

The disclosed system produces useful ultrasound images in tissue while operating within eye and skin safety limits. Additionally, PA mechanisms can produce the full compilation of ultrasonic waves including compressional, shear, Rayleigh, and Love wave components. Using information from these various wave types yields not only anatomical images in the body, but can also provide elastic property distributions, in-situ that are useful for several emerging fields in medicine.

The system disclosed herein induces a probing ultrasonic wave initiated with an optical short pulse that triggers photoacoustic phenomena. This short pulse converts optical energy into mechanical energy via thermal loading, causing the optical absorbing material—human skin or structure surface—to locally deform very rapidly (stress-strain yields a propagating elastic wave) and launch the probing ultrasonic wave that can travel several inches into the body and back. The probing wave reflects off varying internal anatomical features with depth and emerges containing the subject interior image signal. The return signal is then measured at the skin or structure surface with a highly-sensitive laser Doppler vibrometer (LDV). The disclosed system does not require coupling gel or any other substance at the skin or target surface to enhance signal transmission as is the case with contact ultrasonic transducers used in routine medical practice.

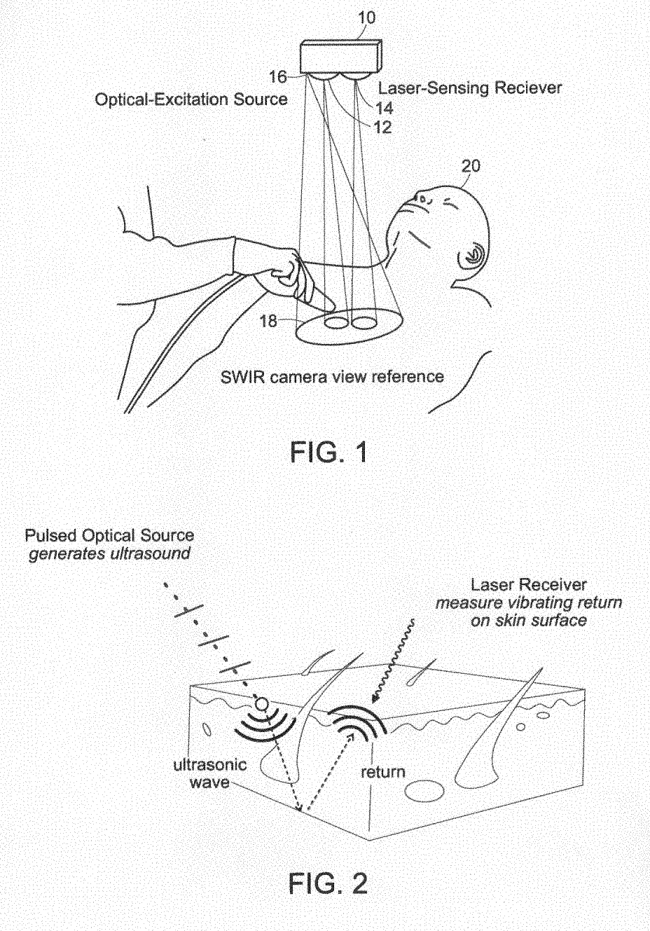

With reference to FIG. 1, apparatus 10 includes an optical excitation source 12, a laser sensing receiver portion 14 and a camera portion 16. In operation, the laser 12 which may be a Q-switched laser illuminates a surface region 18 on a human body 20. The skin is thermally deformed rapidly resulting in a propagating elastic wave into the body 20. The ultrasonic wave travels into the body and is reflected back to the skin where the laser sensing receiver 14 detects the vibration at the surface. The laser sensing receiver 14 is preferably a highly sensitive laser Doppler vibrometer (LDV). The apparatus 10 may also include a data acquisition module for receiving the vibrometer information to create an image.

FIG. 2 illustrates how the system works. A pulsed optical source generates ultrasound through photoacoustic (PA) mechanisms. The laser receiver measures the vibrating return on the skin surface.

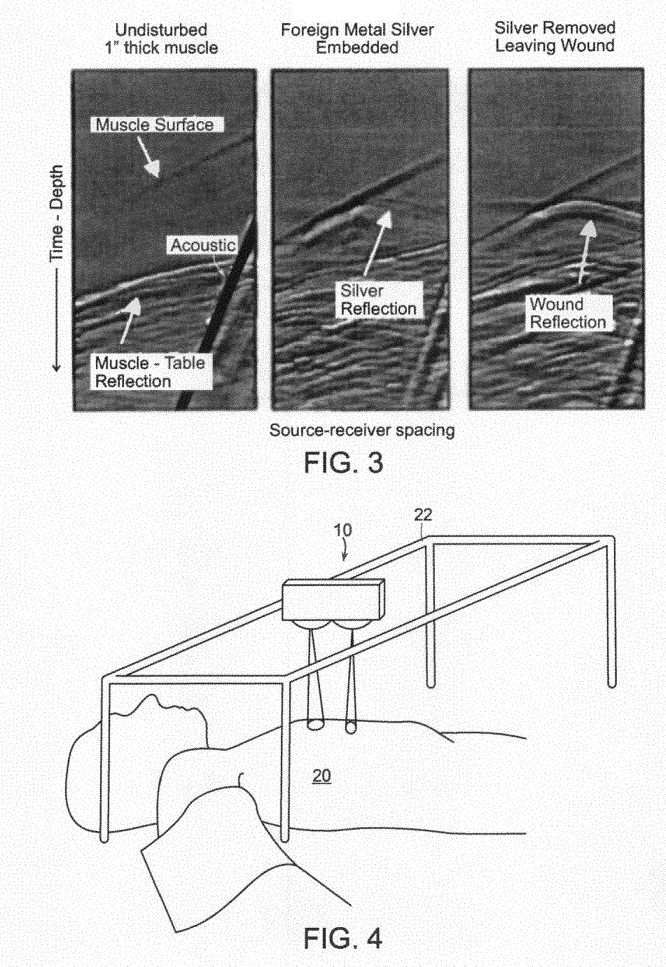

With reference now to FIG. 3, the left side panel shows a one inch thick muscle sample supported on a metal tabletop. A metal sliver is embedded one half inch below the muscle surface without disturbing the muscle surface. Its image is shown in the middle panel of FIG. 3. The rightmost panel in FIG. 3 shows a wound reflection after the metal sliver has been removed. The measurement was made from approximately a one meter standoff from the muscle specimen.

FIG. 4 shows the apparatus 10 supported on an x-y stage which is supported by the frame or stand 22. The stand 22 is useful in military field operations. The components shown in FIG. 4

include an excitation laser to launch ultrasonic waves, a fast steering mirror that directs the excitation laser and a receiving laser vibrometer that measures the ultrasonic return on the patient 10 skin surface. The components also include a shortwave infrared (SWIR) camera 16 to image the skin surface and laser contact points. The apparatus 10 also includes an acquisition system that records, stores and processes the acquired ultrasonic data to form the ultrasonic images. An optical camera can also be used to provide a fixed reference frame when using a simple red or green eye-safe laser. Lidar can also be used to provide a topographical reference of the patient surface that can be updated at a rate of 1 kilohertz to aid in data processing and image processing.

In a preferred embodiment, the Q-switched laser generates short optical pulses of approximately 3 nsec at a 1550 nm optical wavelength. A suitable beam diameter at a skin surface is 1-2 mm. Optical fluence level is 21 mJ/cm². The laser may be a Continuum Panther OPO or a Continuum Minilite YAG Q-switched laser, for example. A suitable Q-switched Doppler vibrometer is a Polytec OFV 5000 LDV or a custom built unit.

Modifications and variations of the invention disclosed herein will be readily apparent to those of skill in the art and it is intended that all such modifications and variations be included within the scope of the appended claims.

What is claimed is:

1. Non-contact ultrasound imaging system comprising:
a pulsed near infrared scanning laser source for illuminating a surface of a structure to generate ultrasonic elastic waves that propagate into the structure;
a laser Doppler vibrometer to measure vibration of the surface from the propagating ultrasonic waves in the structure; and
a data acquisition module for processing data from the vibrometer to construct an image of the structure.

2. The system of claim 1 further including a camera to record locations of illuminating and vibrometer beam locations.

3. The system of claim 1 wherein the pulsed near infrared scanning laser is a Q-switched laser.

4. The system of claim 3 wherein the Q-switched laser operates at a 1-10 kilohertz framerate.

5. The system of claim 1 wherein the structure is a biological entity and the surface is skin.

6. The system of claim 1 wherein the system components are supported by an x-y translation stage mounted on a stand that fits over the structure.

7. The system of claim 1 wherein the laser source operates at approximately a 1500 nm wavelength.
